(19) United States
(12) Patent Application Publication
(10) Pub. No.: US 2026/0355200 A1
(43) Pub. Date: Nov. 12, 2026

(54) MINI-TABLET DOSAGE FORM OF A VIRAL TERMINASE INHIBITOR AND USES THEREOF

(71) Applicant: Merck Sharp & Dohme LLC, Rahway, NJ (US)

(72) Inventors: Cory Bottone, Clinton, NJ (US); Andrew J. Gmitter, Collegeville, PA (US); Ashley R. Johnson, Ambler, PA (US); Eric A. Kemp, Souderton, PA (US); Feng Li, Dresher, PA (US); Fochive Njikam, Dresher, PA (US); Michael H. Wang, Harleysville, PA (US)

(73) Assignee: Merck Sharp & Dohme LLC, Rahway, NJ (US)

(21) Appl. No.: 19/739,034

(22) Filed: Jul. 13, 2026

Related U.S. Application Data
(63) Continuation of application No. 18/256,943, filed on Dec. 14, 2021, now abandoned.
(60) Provisional application No. 63/126,148, filed on Dec. 16, 2020.

(51) Int. Cl. A61K 31/517; A61K 9/20; A61K 9/28

(52) U.S. Cl. CPC A61K 31/517; A61K 9/2009; A61K 9/2013; A61K 9/2027; A61K 9/2054; A61K 9/2095; A61K 9/2866; A61K 9/2893

(57) ABSTRACT

The present invention relates to oral dosage forms comprising letermovir and, in particular, to compressed mini-tablets comprising letermovir. The invention also relates to methods of using the oral dosage forms for the treatment, prevention, or prophylaxis of HCMV in a patient. In addition, the invention also provides methods for making the mini-tablets of the invention.

### CROSS-REFERENCE TO RELATED APPLICATIONS

This application is a continuation of U.S. patent application Ser. No. 18/256,943, filed Jun. 12, 2023, which is a National Stage Entry of PCT/US2021/063179 filed Dec. 14, 01, which claims priority benefit of U.S. Provisional Patent Application Ser. No. 63/126,148 filed Dec. 16, 2020, the entire contents of which are incorporated by reference herein.

### FIELD OF THE INVENTION

The present invention relates to compressed mini-tablets comprising a viral terminase inhibitor. The invention also relates to methods of using the mini-tablets for the treatment, prevention, or prophylaxis of human cytomegalovirus (HCMV) infection in a patient.

### BACKGROUND OF THE INVENTION

The goal of any drug delivery system is to provide a therapeutic amount of drug to the proper site in the body to achieve, and then maintain the desired drug concentration. The most convenient and commonly employed route of drug delivery has historically been by solid oral dosage forms, particularly tablets, and capsules. However, conventional tablets and capsules are limited by their rigid dose content. Furthermore, difficulty swallowing tablets and capsules is a problem for many patients, including pediatric and elderly patients, and can lead to a variety of adverse events, and patient noncompliance with treatment regimens.

Mini-tablets represent an advance in solid dosage form design, with the main goal of overcoming common therapeutic obstacles. Mini-tablets are multiple unit dosage forms and offer several advantages over standard tablets and capsules, such as ease of manufacture, requirement for less coating materials, and considerable flexibility during their formulation development. Mini-tablets are particularly useful for administering to pediatric, and elderly patients, as they are easy to swallow.

Letermovir ((4S)-2-{8-Fluoro-2-[4-(3-methoxyphenyl)piperazin-1-yl]-3-[2-methoxy-5(trifluoromethyl)phenyl]-3,4-dihydroquinazolin-4-yl}acetic acid) is a novel agent that represents a new class of non-nucleoside CMV inhibitors, the 3,4 dihydro-quinazoline-4-yl-acetic acid derivatives. Letermovir was approved by the United States Food and Drug Administration (FDA) for the prophylaxis of CMV infection and disease in adult CMV-seropositive allo-HCT recipients in November 2017. Unlike other anti-CMV therapies that are currently available, letermovir has activity in the late stages of viral replication rather than against the viral DNA polymerase.

The structure of letermovir is as follows:

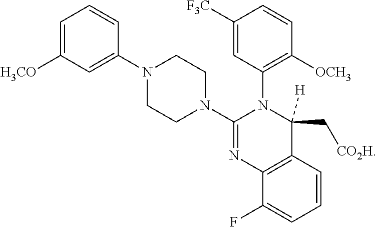 [SMILES: COc1cccc(N2CCN(C3=Nc4c(F)cccc4[C@H](CC(=O)O)N3c3cc(C(F)(F)F)ccc3OC)CC2)c1]

Letermovir is described, for example, in U.S. Pat. Nos. RE46791 and 8,513,255.

U.S. Pat. Nos. 9,890,128 and 10,392,353 disclose methods that can be used to prepare letermovir.

There remains a need for novel letermovir oral dosage forms that are more amenable to patient populations that are averse to standard oral dosage forms and provide the ability to dose patient populations that cannot swallow intact tablets. This invention addresses that need.

### SUMMARY OF THE INVENTION

In one aspect, the present invention provides a dosage form comprising:
(a) about 1 to about 5 mg of letermovir; and
(b) a pharmaceutically acceptable carrier.

In one embodiment is provided a tablet comprising:
(a) about 1 to about 5 mg of letermovir; and
(b) a pharmaceutically acceptable carrier;
wherein the tablet has a diameter of from about 1 to about 4 mm, and a total weight of from about 2 to about 15 mg.

In another particular embodiment are provided mini-tablets (the “mini-tablets of letermovir” or the “mini-tablets”) comprising:
(a) about 1 to about 5 mg of letermovir; and
(b) a pharmaceutically acceptable carrier;
   wherein the mini-tablet has a diameter of from about 1 to about 4 mm, and a total weight of from about 2 to about 15 mg.

In another aspect, the present invention provides a method for the treatment, prevention, or prophylaxis of human cytomegalovirus (HCMV) in a patient, said method comprising administering to said patient one or more mini-tablets of letermovir.

The mini-tablets of letermovir can be useful, for example, for inhibiting HCMV viral replication activity, and for the treatment, prevention, or prophylaxis of HCMV infection infection in a patient. Without being bound by any specific theory, it is believed that letermovir inhibits HCMV viral replication by inhibiting HCMV terminase.

The details of the invention are set forth in the accompanying detailed description below.

Although any methods and materials similar to those described herein can be used in the practice or testing of the present invention, illustrative methods and materials are now described. Other embodiments, aspects and features of the present invention are either further described in or will be apparent from the ensuing description, examples and appended claims.

### DETAILED DESCRIPTION OF THE INVENTION

The present invention relates to novel oral dosage forms of letermovir, including mini-tablets, methods of using such dosage forms of letermovir for the treatment, prevention, or prophylaxis of HCMV infection in a patient, and methods for making the dosage forms of the present invention.

### DEFINITIONS AND ABBREVIATIONS

The terms used herein have their ordinary meaning and the meaning of such terms is independent at each occurrence thereof. That notwithstanding, and except where stated otherwise, the following definitions apply throughout the specification and claims. Chemical names, common names, and chemical structures may be used interchangeably to describe the same structure. If a chemical compound is referred to using both a chemical structure and a chemical name, and an ambiguity exists between the structure and the name, the structure predominates. These definitions apply regardless of whether a term is used by itself or in combination with other terms, unless otherwise indicated.

As used herein, and throughout this disclosure, the following terms, unless otherwise indicated, shall be understood to have the following meanings:

A “mini-tablet” is a tablet having a diameter of from about 1 to about 4 mm, and a total weight of from about 2 to about 15 mg.

A “patient” is a human. In one embodiment, the patient is an adult CMV-seropositive recipients [R+] of an allogeneic hematopoietic stem cell transplant (HSCT). In another embodiment, a patient is a pediatric CMV-seropositive recipients [R+] of an allogeneic hematopoietic stem cell transplant (HSCT). In one embodiment, the patient is an adult CMV-seropositive recipients [R+] of an solid organ transplant (SOT). In another embodiment, the patient is an pediatric CMV-seropositive recipients [R+] of an solid organ transplant (SOT). The term “dosage form” refers to a pharmaceutical product comprising an active pharmaceutical ingredient (API), such as letermovir, wherein the pharmaceutical product is in the form suitable for administration. The dosage form comprises a mixture of active drug component(s) and nondrug component(s) (excipient(s)), along with other non-reusable material that may not be considered either ingredient or packaging (such as a capsule shell, for example).

The term “dosage form” also refers to a chemical formulation comprising a mini-tablet of letermovir and any blends involved, without considering its ultimate configuration as a consumable tablet. A dosage forms may exist in several types. Non-limiting dosage forms include tablets, capsules, sachets, and stick packs. A dosage form is administered orally. In embodiment, the dosage form is a mini-tablet of letermovir.

The term “effective amount” as used herein, refers to an amount of letermovir and/or an additional therapeutic agent, that is effective in inhibiting HCMV replication and preferably in producing the desired therapeutic, ameliorative, inhibitory, prophylactic, or preventative effect when administered to a patient. In the combination therapies of the present invention, an effective amount can refer to each individual agent or to the combination as a whole, wherein the amounts of all agents administered are together effective.

The term “preventing,” as used herein with respect to an HCMV viral infection or HCMV-virus related disorder, refers to reducing the likelihood of HCMV infection.

The term “prophylaxis,” as used herein with respect to an HCMV viral infection or HCMV-virus related disorder, refers to reducing the likelihood of latent HCMV virus becoming reactivated in a patient. In one embodiment, the term “prophylaxis” refers to prophylaxis of CMV infection and disease in adult CMV-seropositive recipients [R+] of an allogeneic hematopoietic stem cell transplant (HSCT). In one embodiment, the term “prophylaxis” refers to prophylaxis of CMV infection and disease in pediatric CMV-seropositive recipients [R+] of an allogeneic hematopoietic stem cell transplant (HSCT). In one embodiment, the term “prophylaxis” refers to prophylaxis of CMV infection and disease in adult CMV-seropositive recipients [R+] of solid organ transplant (SOT). In one embodiment, the term “prophylaxis” refers to prophylaxis of CMV infection and disease in pediatric CMV-seropositive recipients [R+] of solid organ transplant (SOT).

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization, and the like), in sufficient purity to be characterizable by standard analytical techniques described herein or well-known to the skilled artisan.

It should also be noted that any carbon as well as any heteroatom with unsatisfied valences in the text, schemes, examples and tables herein is assumed to have the sufficient number of hydrogen atom(s) to satisfy the valences.

As used herein, the term “composition” is intended to encompass a product comprising the specified ingredients in the specified amounts, as well as any product (such as a mini-tablet of letermovir) which results from combination of the specified ingredients in the specified amounts.

### The Mini-Tablets of Letermovir

Provided is a dosage form comprising:
(a) about 1 to about 5 mg of letermovir; and
(b) a pharmaceutically acceptable carrier.

In one embodiment is provided a tablet comprising:
(a) about 1 to about 5 mg of letermovir; and
(b) a pharmaceutically acceptable carrier;
wherein the tablet has a diameter of from about 1 to about 4 mm, and a total weight of from about 2 to about 15 mg.

In another embodiment is provided a tablet comprising:
(a) 1 to 5 mg of letermovir; and
(b) a pharmaceutically acceptable carrier;
wherein the tablet has a diameter of from 1 to 4 mm, and a total weight of from 2 to 15 mg.

In another embodiment there is provided a mini-tablet comprising:
(a) about 1 to about 5 mg of letermovir; and
(b) a pharmaceutically acceptable carrier;
   wherein the mini-tablet has a diameter of from about 1 to about 4 mm, and a total weight of from about 2 to about 15 mg.

In one embodiment are provided mini tablets of letermovir comprising:
(a) 1 to 5 mg of letermovir; and
(b) a pharmaceutically acceptable carrier;
   wherein the mini-tablet has a diameter of from 1 to 4 mm, and a total weight of from 2 to 15 mg

In one embodiment, the amount of letermovir in the mini-tablet is 5 mg.

In another embodiment, the amount of letermovir in the mini-tablet is 4 mg.

In another embodiment, the amount of letermovir in the mini-tablet is 3 mg.

In still another embodiment, the amount of letermovir in the mini-tablet is 2.5 mg.

In another embodiment, the amount of letermovir in the mini-tablet is 2 mg.

In yet another embodiment, the amount of letermovir in the mini-tablet is 1.5 mg.

In another embodiment, the amount of letermovir in the mini-tablet is 1 mg.

In one embodiment, the mini-tablet has a diameter of about 2.5 mm.

In another embodiment, the mini-tablet has a diameter of about 3.0 mm.

In another embodiment, the mini-tablet has a diameter of about 3.5 mm.

In still another embodiment, the mini-tablet has a diameter of about 4.0 mm.

In another embodiment, the mini-tablet has a diameter of less than or equal to 2.5 mm.

In another embodiment, the mini-tablet has a diameter of about 2 mm.

In another embodiment, the mini-tablet has a diameter of about 1.5 mm.

In one embodiment, the total weight of the mini-tablet is from 4 mg to 12 mg.

In another embodiment, the total weight of the mini-tablet is from 6 mg to 9 mg.

In another embodiment, the total weight of the mini-tablet is from 7 mg to 8 mg.

In one embodiment, the amount of letermovir in the mini-tablet is about 2.5 mg, the total weight of the mini-tablet is from 6 mg to 9 mg, and the diameter of the mini-tablet is about 2 mm.

In one embodiment, the mini-tablet of letermovir further comprises a diluent, a glidant, a binder, a disintegrant, and a lubricant.

In another embodiment, the mini-tablet of letermovir further comprises a diluent, a glidant, a binder, a disintegrant, a lubricant, and a film coat blend.

In one embodiment, the mini-tablet of letermovir further comprises a diluent which is microcrystalline cellulose, a glidant which is silicon dioxide, a binder which is povidone, a disintegrant which is croscarmellose sodium, and a lubricant which is magnesium stearate.

In another embodiment, the mini-tablet of letermovir further comprises a diluent which is microcrystalline cellulose, a glidant which is silicon dioxide, a binder which is povidone, a disintegrant which is croscarmellose sodium, a lubricant which is magnesium stearate, and a film coat blend which is Opadry II.

In one embodiment, the tablet of letermovir comprises:
(a) 2.5 mg letermovir;
(b) about 3.011 mg microcrystalline cellulose;
(c) about 0.2081 mg povidone;
(d) about 0.3125 mg croscarmellose sodium;
(e) about 0.125 mg silicon dioxide;
(f) about 0.09375 mg magnesium stearate; and
(g) about 0.9375 mg Opadry II.

In one embodiment, the mini-tablet of letermovir comprises:
(a) 2.5 mg letermovir;
(b) about 3.011 mg microcrystalline cellulose;
(c) about 0.2081 mg povidone;
(d) about 0.3125 mg croscarmellose sodium;
(e) about 0.125 mg silicon dioxide;
(f) about 0.09375 mg magnesium stearate; and
(g) about 0.9375 mg Opadry II.

In one embodiment, the letermovir present in the mini-tablets of the present invention is in substantially purified form.

In one aspect, the present invention relates to processes for preparing mini-tablets of letermovir. The mini-tablets of letermovir may be prepared using methods known to one skilled in the art of pharmaceutical formulation. Methods useful for making the mini-tablets of letermovir are set forth herein. Alternative methods for making the mini-tablets of letermovir are well known to one skilled in the art of pharmaceutical formulation.

Accordingly, in one embodiment, the present invention provides a process for making a mini-tablet of letermovir, comprising the following sequential steps:
(a) blending a diluent, a glidant, letermovir, a binder, and a disintegrant;
(b) adding a lubricant to the blend obtained from step a, and roller compacting the resulting mixture;
(c) adding additional lubricant to the roller-compacted material obtained from step b, and compressing the resulting mixture into a mini-tablet;
(d) optionally film coating the mini-tablet obtained from step c; and
(e) packaging the film-coated tablet obtained from step c or d.

In one embodiment, the film coated process of step d is performed.

In another embodiment, the film coated process of step d is performed in order to mask the taste of the mini-tablet of letermovir.

In still another embodiment, the film-coating process of step d is performed using fluid bed drying.

In another embodiment, the film coated process of step d is not performed.

In one embodiment, the process of the present invention further comprises the step of de-dusting the tablet obtained from step c, prior to performing the film coating of step d.

In one embodiment, for the process of the present invention, the diluent is microcrystalline cellulose, the glidant is silicon dioxide, the binder is povidone, the disintegrant is croscarmellose sodium, and the lubricant is magnesium stearate.

In another embodiment, for the process of the present invention, the diluent is microcrystalline cellulose, the glidant is silicon dioxide, the binder is povidone, the disintegrant is croscarmellose sodium, the lubricant is magnesium stearate, and the film coat material is Opadry II.
(a) A method of inhibiting HCMV replication in a subject in need thereof which comprises administering to the subject an effective amount of one or more mini-tablets of letermovir.
(b) A method for the treatment, prevention, or prophylaxis of HCMV infection and/or reducing the likelihood or severity of symptoms of HCMV infection in a subject in need thereof, which comprises administering to the subject an effective amount of one or more mini-tablets of letermovir.
(c) The method of (a) or (b), wherein the one or more mini-tablets of letermovir is administered in combination with an effective amount of at least one second therapeutic agent selected from the group consisting of HCMV antiviral agents.

The present invention also includes mini-tablets of letermovir for use (i) in, (ii) as a medicament for, or (iii) in the preparation of a medicament for: (a) medicine; (b) inhibiting HCMV replication, (c) treating HCMV infection and/or reducing the likelihood or severity of symptoms of HCMV infection, (d) prevention of HCMV infection, and (e) prophylaxis of HCMV infection. In these uses, the mini-tablets of letermovir can optionally be employed in combination with one or more additional therapeutic agents selected from HCMV antiviral agents, and other anti-infective agents.

It is further to be understood that the embodiments of compositions and methods provided as (a) through (c) above are understood to include all embodiments of the compounds, including such embodiments as result from combinations of embodiments.

### Pharmaceutically Acceptable Carriers

The mini-tablets of letermovir comprise a pharmaceutically acceptable carrier, which consists of one or more excipients, including, but not limited to diluents, glidants, binders, disintegrants and lubricants.

Non-limiting examples of diluents useful in the present invention include microcrystalline cellulose, anhydrous lactose, and lactose monohydrate, and sugar alcohols such as sorbitol, xylitol and mannitol. In one embodiment, the diluent is microcrystalline cellulose. In one embodiment, the diluent is present in an amount of from 35% to 45% by weight of the mini-tablet. In another embodiment, the diluent is present in an amount of from 41% to 42% by weight of the mini-tablet.

Non-limiting examples of glidants useful in the present invention include silicon dioxide, talc, silica, stearates (magnesium, calcium, and sodium), stearic acid, sodium stearyl fumarate, and sodium lauryl sulfate. In one embodiment, the glidant is silicon dioxide. In one embodiment, the glidant is present in an amount of from 1% to 3% by weight of the mini-tablet. In another embodiment, the diluent is present in an amount of from 1.5% to 2.0% by weight of the mini-tablet.

Non-limiting examples of binders useful in the present invention include povidone, cellulose, methyl cellulose, polyvinylpyrrolidone and polyethylene glycol. In one embodiment, the binder is povidone. In one embodiment, the glidant is silicon dioxide. In one embodiment, the binder is present in an amount of from 2% to 4% by weight of the mini-tablet. In another embodiment, the diluent is present in an amount of from 2.5% to 3.0% by weight of the mini-tablet.

Non-limiting examples of disintegrants useful in the present invention include croscarmellose sodium, sodium starch glycolate, and crosslinked polymers (such as crospovidone). In one embodiment, the disintegrant is croscarmellose sodium. In one embodiment, the disintegrant is present in an amount of from 3% to 5% by weight of the mini-tablet. In another embodiment, the disintegrant is present in an amount of from 4% to 4.5% by weight of the mini-tablet.

Non-limiting examples of lubricants useful in the present invention include magnesium stearate, talc, silica, vegetable stearin, sodium stearyl fumarate, and stearic acid. In one embodiment, the lubricant is magnesium stearate. In one embodiment, the lubricant is present in an amount of from 0.5% to 2% by weight of the mini-tablet. In another embodiment, the lubricant is present in an amount of from 1.0% to 1.5% by weight of the mini-tablet.

### Film Coat

In one aspect of the present invention, the mini-tablets of letermovir can be film-coated. The film coating may be used for various purposes, including, but not limited to, masking the taste of the mini-tablet of letermovir, improving the appearance of the mini-tablet of letermovir, to stabilize the mini-tablet of letermovir, to modify or delay the release of letermovir, or to facilitate swallowing of the mini-tablet of letermovir.

In one embodiment, the mini-tablet is film-coated. In one embodiment, the film coat blend is Opadry II. In one embodiment, the film coat blend is present in an amount of from 10% to 20% by weight of the mini-tablet. In another embodiment, the lubricant is present in an amount of from 12% to 14% by weight of the mini-tablet.

In another embodiment, the mini-tablet is not film-coated.

### Uses of the Mini-Tablets of Letermovir

### Treatment, Prevention or Prophylaxis of HCMV Infection

The mini-tablets of letermovir are useful in the inhibition of HCMV (e.g., HCMV terminase), the treatment or prevention of HCMV infection and/or reduction of the likelihood or severity of symptoms of HCMV infection and the inhibition of HCMV viral replication and/or HCMV viral production in a cell-based system. Mini-Tablets of letermovir are also useful in prophylaxis of HCMV infection following hematopoietic stem cell transplant, or solid organ transplant in a patient.

In one embodiment, the mini-tablets of letermovir are administered to a patient for prophylaxis of HCMV infection following hematopoietic stem cell transplant.

In another embodiment, the mini-tablets of letermovir are administered to a patient for prophylaxis of HCMV infection following solid organ transplant.

In another embodiment, the mini-tablets of letermovir are administered to a patient for prophylaxis of HCMV infection following kidney transplant.

Accordingly, in one embodiment, the invention provides methods for the treatment, prevention, or propylaxis of HCMV infection in a patient, the methods comprising administering to the patient an effective amount of one or more mini-tablets of letermovir. In one embodiment, the methods comprise administering to the patient four or more mini-tablets of letermovir. In a specific embodiment, the amount administered is effective to treat or prevent infection by HCMV in the patient. In another specific embodiment, the amount administered is effective to inhibit HCMV viral replication and/or viral production in the patient.

### Compositions and Administration

The present invention provides mini-tablets of letermovir comprising letermovir, and a pharmaceutically acceptable carrier. In the mini-tablets, and methods of the present invention, the active ingredients will typically be administered in admixture with suitable carrier materials suitably selected with respect to the intended form of administration, i.e., oral tablets, capsules, sachets, stick packs, and the like, and consistent with conventional pharmaceutical practices. For example, for oral administration in the form of tablets or capsules, the active drug component may be combined with any oral non-toxic pharmaceutically acceptable inert carrier. Solid form preparations include tablets, capsules, sachets and suppositories. The mini-tablets of letermovir may be comprised of from about 6% to about 70% percent letermovir, by weight percentage. The mini-tablets of letermovir are useful as solid dosage forms suitable for oral administration.

Moreover, when desired or needed, additional suitable binders, lubricants, disintegrating agents and coloring agents may also be incorporated in the mixture. Sweetening and flavoring agents and preservatives may also be included where appropriate.

Additionally, the mini-tablets of letermovir of the present invention may be formulated in sustained release form to provide the rate controlled release of any one or more of the components or active ingredients to optimize therapeutic effects, i.e., antiviral activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

In one embodiment, the mini-tablet of letermovir is administered orally.

In still another embodiment, the mini-tablet of letermovir is administered sublingually.

In one embodiment, a pharmaceutical preparation comprising one or more mini-tablets of letermovir is in unit dosage form. In such form, the preparation is subdivided into unit doses containing effective amounts of the active components. In one embodiment, four or more mini-tablets of letermovir are administered to a patient to achieve the desired dose of letermovir.

In one embodiment, the present invention provides a method for the treatment, prevention or prophylaxis of human cytomegalovirus in a patient, comprising orally administering to the patient four or more of the mini-tablets of the present invention.

In another embodiment, the present invention provides a method for the treatment of human cytomegalovirus in a patient, comprising orally administering to the patient four or more of the mini-tablets of the present invention.

In another embodiment, the present invention provides a method for the prevention of human cytomegalovirus in a patient, comprising orally administering to the patient four or more of the mini-tablets of the present invention.

In still another embodiment, the present invention provides a method for prophylaxis of human cytomegalovirus in a patient, comprising orally administering to the patient four or more of the mini-tablets of the present invention.

In one embodiment, four or more mini-tablets are packaged in a capsule, a sachet, or a stick-pack.

Mini-tablets of letermovir can be prepared using techniques such as conventional mixing, granulating or coating methods; and by using solid dispersion based upon the guidance provided herein. In one embodiment, the mini-tablets of letermovir can contain from about 6% to about 70% of letermovir by weight. In various embodiments, the mini-tablets of letermovir can contain, in one embodiment, from about 10% to about 60%, from about 20-50%, and from about 30-40% of letermovir by weight. In one embodiment, the mini-tablets of letermovir contain from 32-36% letermovir by weight.

The quantity of letermovir in a unit dose of preparation may be varied or adjusted from 10 mg to 480 mg. In various embodiments, the quantity is from 10 mg to 240 mg, 10 mg to 100 mg, 10 mg to about 50 mg, 50 mg, 40 mg, 30 mg, 20 mg, and 10 mg.

The doses and dosage regimen of the other agents used in the combination therapies of the present invention for the treatment or prevention of HCMV infection can be determined by the attending clinician, taking into consideration the approved doses and dosage regimen in the package insert; the age, sex and general health of the patient; and the type and severity of the viral infection or related disease or disorder. When administered in combination, the mini-tablets of letermovir and the other agent(s) can be administered simultaneously (i.e., in the same composition or in separate compositions one right after the other) or sequentially. This is particularly useful when the components of the combination are given on different dosing schedules, e.g., one component is administered once daily and another component is administered every six hours, or when the preferred pharmaceutical compositions are different, e.g., one is a tablet and one is a capsule. A kit comprising the separate dosage forms can therefore be advantageous.

Generally, a total daily dosage of letermovir alone, when administered as combination therapy, can range from about 1 to about 480 mg per day, although variations will necessarily occur depending on the target of therapy, the patient and the route of administration. In one embodiment, the dosage is from about 10 to about 240 mg/day, administered in a single dose or in 2-4 divided doses. In another embodiment, the dosage is from about 10 to about 100 mg/day, administered in a single dose or in 2-4 divided doses. In still another embodiment, the dosage is from about 10 to about 50 mg/day, administered in a single dose or in 2-4 divided doses.

In one embodiment, the total daily dose is administered once daily. In another embodiment, the total daily dose is administered once daily for 100 days post hematopoietic stem cell transplant (HSCT). In another embodiment, the total daily dose is administered once daily for 100 days post solid-organ transplant (SOT).

For convenience, the total daily dosage may be divided, and administered in portions during the day if desired. In one embodiment, the daily dosage is administered in one portion.

In another embodiment, the total daily dosage is administered in two divided doses over a 24-hour period. In another embodiment, the total daily dosage is administered in three divided doses over a 24-hour period. In still another embodiment, the total daily dosage is administered in four divided doses over a 24-hour period.

In one embodiment, the total daily dose is administered once daily. In another embodiment, the total daily dose is administered once daily for 100 days post hematopoietic stem cell transplant (HSCT). In another embodiment, the total daily dose is administered once daily for 100 days post solid-organ transplant (SOT).

The amount and frequency of administration of one or more mini-tablets of letermovir will be regulated according to the judgment of the attending clinician considering such factors as age, condition and size of the patient as well as severity of the symptoms being treated. Generally, a total daily dosage of the mini-tablets of letermovir will necessarily occur depending on the age or weight of the patient.

In one embodiment, the patient is an adult patient.

In another embodiment, the patient is a geriatric patient.

In another embodiment, the patient is a patient requiring a gastric or nasogastric tube.

In still another embodiment, the patient is a pediatric patient.

In another embodiment, the patient is an adolescent aged 12 years through 21 years.

In yet another embodiment, the patient is child aged 2 years to less than 12 years.

In one embodiment, the patient is an infant less than 2 years old.

In another embodiment, the patient is an infant less than 1 year old.

In another embodiment, the patient is an infant less than 6 months old.

In still embodiment, the patient is a neonate.

### Combination Therapy

In another embodiment, the present methods for treating or preventing HCMV infection can further comprise the administration of one or more additional therapeutic agents that are other than letermovir.

In one embodiment, the additional therapeutic agent is an antiviral agent.

In another embodiment, the additional therapeutic agent is an anti-HCMV agent.

Accordingly, in one embodiment, the present invention provides methods for treating a viral infection in a patient, the method comprising administering to the patient: (i) a mini-tablet of letermovir, or a pharmaceutically acceptable salt thereof, and (ii) at least one additional therapeutic agent that is other than letermovir, wherein the amounts administered are together effective for the treatment, prevention, or prophylaxis of HCMV infection in a patient.

When administering a combination therapy of the invention to a patient, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, the mini-tablet of letermovir and an additional therapeutic agent may be present in fixed amounts (dosage amounts) in a single dosage unit (e.g., a capsule, a tablet and the like).

In one embodiment, the mini-tablet of letermovir is administered during a time when the additional therapeutic agent(s) exert their prophylactic or therapeutic effect, or vice versa.

In another embodiment, the mini-tablet of letermovir and the additional therapeutic agent(s) are administered in doses commonly employed when such agents are used as monotherapy for the treatment, prevention, or prophylaxis of HCMV infection.

In another embodiment, the mini-tablet of letermovir and the additional therapeutic agent(s) are administered in doses lower than the doses commonly employed when such agents are used as monotherapy for the treatment, prevention, or prophylaxis of HCMV infection.

In still another embodiment, the mini-tablet of letermovir and the additional therapeutic agent(s) act synergistically and are administered in doses lower than the doses commonly employed when such agents are used as monotherapy for the treatment, prevention, or prophylaxis of HCMV infection.

In one embodiment, the additional therapeutic agent(s) is present as a component of the mini-tablet of letermovir. This composition is suitable for oral administration.

The mini-tablet of letermovir and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy.

In one embodiment, the administration of the mini-tablet of letermovir and the additional therapeutic agent(s) may inhibit the resistance of HCMV to these agents.

Non-limiting examples of additional therapeutic agents useful in the present compositions and methods include immunoglobulins, antiviral agents, purine nucleoside inhibitors, and any agent useful for treating or preventing HCMV infection.

In one embodiment, the additional therapeutic agent(s) are selected from ganciclovir, valacyclovir, valganciclovir, and cytomegalovirus immune globulin.

In one embodiment, the additional therapeutic agent is a ganciclovir.

In another embodiment, the additional therapeutic agent is valacyclovir.

In still another embodiment, the additional therapeutic agent is vanganciclovir.

In yet another embodiment, the additional therapeutic agent is cytomegalovirus immune globulin.

In another embodiment, the additional therapeutic agent is an agent useful for treating or preventing HCMV infection.

In one embodiment, the mini-tablet of letermovir is administered with one or more additional therapeutic agents selected from include immunoglobulins, antiviral agents, purine nucleoside inhibitors, and any agent useful for treating or preventing HCMV infection. The combination therapies can include any combination of these additional therapeutic agents.

In another embodiment, the mini-tablet of letermovir is administered with one or more additional therapeutic agents selected from ganciclovir, valacyclovir, valganciclovir, and cytomegalovirus immune globulin. The combination therapies can include any combination of these additional therapeutic agents.

In another embodiment, the mini-tablet of letermovir is administered with one additional therapeutic agent selected from immunoglobulins, antiviral agents, purine nucleoside inhibitors, and any agent useful for treating or preventing HCMV infection.

In still another embodiment, the mini-tablet of letermovir is administered with two additional therapeutic agents selected from immunoglobulins, antiviral agents, purine nucleoside inhibitors, and any agent useful for treating or preventing HCMV infection.

In another embodiment, the mini-tablet of letermovir is administered with one additional therapeutic agent selected from ganciclovir, valacyclovir, valganciclovir, and cytomegalovirus immune globulin.

In still another embodiment, the mini-tablet of letermovir is administered with two additional therapeutic agents selected from ganciclovir, valacyclovir, valganciclovir, and cytomegalovirus immune globulin.

### Kits

In one aspect, the present invention provides a kit comprising a therapeutically effective amount of one or more mini-tablets of letermovir.

In another aspect the present invention provides a kit comprising an amount of one or more mini-tablets of letermovir, and an amount of at least one additional therapeutic agent listed above, wherein the amounts of the two or more active ingredients result in a desired therapeutic effect. In one embodiment, the mini-tablet of letermovir and the one or more additional therapeutic agents are provided in the same container. In one embodiment, the mini-tablet of letermovir and the one or more additional therapeutic agents are provided in separate containers.

### EXAMPLES

### Example 1

### Screening of Raw Materials

Prior to charging into the blender vessel, some of the raw materials were screened for better mixing, as follows:
Silicon dioxide was manually mixed with microcrystalline cellulose, and the mixture was co-sieved through a 30 mesh screen using a manual or sieve shaker.
Magnesium stearate was sieved through a 60 mesh screen by manual push-though.

### Example 2

### Blending and Lubrication Steps

The making of the formulation of illustrative mini-tablets of the present invention employed diffusion mixing mechanisms for blending and lubrication. The equipment used was a diffusion mixer (i.e., such as a blender made by Bohle® or P-K Blendmaster®). The critical processing parameters are the fill level and the number of revolutions, with both parameters considered scale-independent. The conditions and the parameters of the blending and lubrication steps used to make the formulation of illustrative mini-tablets of the present invention are summarized in the table below:

| Unit Operation | Equipment and Scales | Range of Key Parameters |
|---|---|---|
| Blending and | Bohle ® or PK ® blender; | Fill level: 0.1-0.25 kg/L |
| lubrication | 2.5 to 100 L vessel; | (~25-75% fill volume); |
| | 300 g to 22 kg | No. of revolution: 240 or |
| | | 250 for blending and 120 |
| | | or 125 for lubrication |

### Example 3

### Roller Compaction Step

Roller compaction (RC) was utilized in the process for making the formulation of the mini-tablets of the present invention prior to compression tableting. The formulation was processed on a pilot scale roller compactor (Alexanderwerk WP120 with RFG) at scales ranging from 300 g to 22 kg. A roll pressure of 35-65 bar was employed, with a constant roll gap of 2.0 mm, and a roll speed of 4-8 RPM was used. A wire mesh screen having a mesh size from 0.5 mm to 1.25 mm was used to produce the final granules for tableting.

### Example 4

### Mini-Tablet Compression

Minitablet compression was utilized for manufacturing illustrative mini-tablets of the present invention. The compression was conducted using a Korsch XL100 press having 7-tip tooling, and 18-tip tooling, and a turret speed of 10-30 RPM was utilized for 0.3 kg to 3 kg batch sizes; and a Fette 1200 press with 18-tip tooling, and a turret speed of 20 RPM was used for 8 kg to 22 kg batch sizes. Acceptable minitablets have been produced in a wide range of the main compression force, 8-17 kN (equivalent to ~150-300 MPa compressive stress). Clinical batches have been successfully manufactured at target force (compressive stress) between 11-14 kN (~200-250 MPa). The pre-compression force was kept within 5-10% of the main compression force. The resulting mini-tablets can be administered as is, or film coated using the method described below in Example 5.

### Example 5

### Mini-Tablet Film Coating

Illustrative mini-tablets of the present invention that were made using the method describe in Example 4, were film coated using a Wurster film coating process that was conducted on: (a) a GPCG3 fluid bed dryer (Glatt GMBH, Binzen, Germany) having a 7″ insert for batch sizes of 1.5 kg to 3 kg; (b) a Niro MP1 (GEA Group AG, Dusseldorf, Germany) fluid bed dryer having a 3″ insert for batch sizes of about 500 g; and (c) a MiniGlatt (Glatt GMBH, Binzen, Germany) fluid bed dryer having a 1.5″ insert for batch sizes of about 50 g. The critical processing parameters are summarized in the table below:

| Unit Operation | Equipment and Scales | Range of Key Parameters |
|---|---|---|
| Film coating | MiniGlatt with 1.5″ | Product temp: 50-60 °C; |
| | insert for ~50 g; | Spray rate: 8-32 g/min; |
| | Niro MP1 with 3″ | Air flow: 120-170 SCFM |
| | insert for ~500 g; | |
| | GPCG3 with 7″ | |
| | insert for 1.5-3 kg | |

1. A mini-tablet comprising:
(a) about 1 to about 5 mg of letermovir; and
(b) a pharmaceutically acceptable carrier;
wherein the mini-tablet has a diameter of from about 1 to about 4 mm, and a total weight of from about 2 to about 15 mg.

2. The mini-tablet of claim 1, wherein the amount of letermovir in the mini-tablet is 2.5 mg.

3. The mini-tablet of claim 1, wherein the amount of letermovir in the mini-tablet is 2.0 mg.

4. The mini-tablet of claim 1, having a diameter of about 2.5 mm.

5. The mini-tablet of claim 1, having a diameter of about 2 mm.

6. The mini-tablet of claim 1, wherein the total weight of the mini-tablet is from about 4 mg to about 12 mg.

7. The mini-tablet of claim 6, wherein the total weight of the mini-tablet is from about 6 mg to about 9 mg.

8. The mini-tablet of claim 1, wherein the amount of letermovir is 2.5 mg, the total weight of the mini-tablet is from about 6 mg to about 9 mg, and the diameter of the mini-tablet is about 2 mm.

9. The mini-tablet of claim 1, comprising a diluent, a glidant, a binder, a disintegrant and a lubricant.

10. The mini-tablet of claim 9, wherein the diluent is microcrystalline cellulose, the glidant is silicon dioxide, the binder is povidone, the disintegrant is croscarmellose sodium, and the lubricant is magnesium stearate.

11. The mini-tablet of claim 10, comprising the following:
(a) 2.5 mg letermovir;
(b) about 3 mg microcrystalline cellulose;
(c) about 0.2 g povidone;
(d) about 0.3125 mg croscarmellose sodium;
(e) about 0.125 mg silicon dioxide;
(f) about 0.09375 mg magnesium stearate; and
(g) about 0.9375 mg film coat blend.

12. The mini-tablet of claim 1, which is film-coated.

13. The mini-tablet of claim 1, which is not film-coated.

14.-23. (canceled)
